(19) United States
(12) Patent Application Publication
(10) Pub. No.: US 2013/0158487 A1
(43) Pub. Date: Jun. 20, 2013

(54) SAFETY INTRAVENOUS NEEDLE SET

(71) Applicant: SUNWELL GLOBAL LIMITED, (US)

(72) Inventor: ZHI-YUN LI, NANTONG CITY (CN)

(73) Assignee: SUNWELL GLOBAL LIMITED

(21) Appl. No.: 13/693,168

(22) Filed: Dec. 4, 2012

(30) Foreign Application Priority Data
Dec. 20, 2011 (TW) 100224058

(51) Int. Cl. A61M 5/32

(52) U.S. Cl. CPC A61M 5/321
USPC 604/263

(57) ABSTRACT

A safety intravenous needle set includes needle hub holding a needle and an infusion tube at two opposite ends thereof, and a safety device, which includes a base block affixed to the needle hub, a recessed guard pivoted to the base block and having a retaining lug located on one of two opposing inner wall thereof. The recessed guard is biasable relative to the needle hub toward the medical needle to let the medical needle be forced over the retaining lug into the inside of the recessed guard and then retaining inside the recessed guard by the retaining lug, avoiding accidental needle stick injury after the service of the safety intravenous needle set.

### BACKGROUND OF THE INVENTION

The present invention is related to a safety intravenous needle set, which is equipped with an anti-piercing safety device to prevent the needle from piercing the healthcare worker accidentally.

A healthcare worker many need to inject a medicine into the body of a patient or to draw blood from a blood vessel of a patient by means of an intravenous needle. However, when handling an intravenous needle during a medical procedure, the healthcare worker may be injured by the needle accidentally.

FIG. 1 illustrates a conventional intravenous needle set. As illustrated, the intravenous needle set 100 comprises a needle cap 10, a medical needle 11, a needle hub 13, and an infusion tube 15.

The needle cap 10 is detachably capped on the medical needle 11 for protection before the use of the intravenous needle set 100. The infusion tube 15 is a fluid passage for intravenous infusion or drawing blood, having its one end connected to the needle hub 13 and its other end provided with a connector 151 for the connection of a feeding tube of an infusion bag, saline bag or blood bag, or a medical device. Further, the needle hub 13 has two wings 22 arranged at two opposite lateral sides thereof.

When using the intravenous needle set 100, the healthcare worker can remove the needle cap 10 from the medical needle 11 to expose the medical needle 11. By means of the wings 12, the healthcare worker can conveniently insert the medical needle 11 into the patient's vein, blood vessel or body, and then secure the wings 12 to the patient's skin by a medical adhesive tape. At this time, the healthcare worker can start an intravenous infusion or blood draw.

After the service of the intravenous needle set 100, the healthcare worker will remove the medical needle 11 from the body of the patient and then cap the needle cap 10 on the medical needle 11 again. However, as the needle cap 10 defines therein a small cap hole, it is difficult to accurately cap the needle cap 10 onto the medical needle 11 at a time, and the healthcare worker may be injured by the medical needle 11 accidentally, leading to a higher risk of virus infection (AIDS, HIV SARS, etc.). Handling a intravenous needle set in this manner is dangerous to healthcare workers.

Therefore, there is a strong demand for a safety intravenous needle set, which enhances safe needle disposal, preventing infections in healthcare workers due to accidental needle injury.

### SUMMARY OF THE PRESENT INVENTION

It is, therefore, the main object of the present invention to provide a safety intravenous needle set, which is equipped with a safety device that can be biased relative to the needle hub to have the medical needle be received inside the safety device after the service of the safety intravenous needle set, avoiding accidental needle stick injury.

It is another object of the present invention to provide a safety intravenous needle set, which enables the medical needle to be accommodated inside a safety device after its service, protecting waste needle handling person against accidental needle stick injury and avoiding a repeat use of the medical needle.

To achieve above objects, the present invention provides a safety intravenous needle set, comprising: a needle hub holding a medical needle at one end thereof and an infusion tube at an opposite end thereof; and a safety device, said safety device comprising a base block, a recessed guard and a pivot, said recessed guard comprises two opposing inner walls, an outer wall and at least one pair of retaining lugs located on said two opposing inner walls, each said pair of retaining lugs defining therebetween a constraint gap, said base block being fixedly mounted at said needle hub, said recessed guard being pivotally connected to said base block by said pivot for allowing said recessed guard to be biased relative to said base block and said needle hub toward said medical needle to let said medical needle be forced through said constraint gap into the inside of said recessed guard.

In one embodiment of the present invention, wherein said recessed guard has a relatively wider top side and a relatively narrower bottom side so that said outer wall defines a bevel face at each of two opposite lateral sides thereof.

In one embodiment of the present invention, wherein said recessed guard comprises an anti-slip structure located on said outer wall.

In one embodiment of the present invention, wherein said anti-slip structure comprises at least one longitudinal rib, at least one transverse rib, at least one raised portion, at least one recessed portion, at least one finger notch and/or at least one finger retaining means.

In one embodiment of the present invention, wherein said needle hub comprises a wing located on at least one of two opposite lateral sides thereof.

In one embodiment of the present invention, wherein each said retaining lug is configured to provide a bevel face, or in the shape of a planar thin sheet member.

In one embodiment of the present invention, wherein said retaining lugs of said safety device are arranged in a staggered manner, defining a longitudinal pitch between each two adjacent said retaining lugs.

In one embodiment of the present invention, wherein said constraint gap has a width equal to or smaller than the diameter of said medical needle.

The present invention further provides a safety intravenous needle set, comprising: a needle hub holding a medical needle at one end thereof and an infusion tube at an opposite end thereof; and a safety device, said safety device comprising a base block, a recessed guard and a pivot, said recessed guard comprises two opposing inner walls, an outer wall and at least one retaining lug located on one said inner wall, said base block being fixedly mounted at said needle hub, said recessed guard being pivotally connected to said base block by said pivot for allowing said recessed guard to be biased relative to said base block and said needle hub toward said medical needle to let said medical needle be forced over said at least one retaining lug into the inside of said recessed guard and then retaining inside said recessed guard by said at least one retaining lug.

In one embodiment of the present invention, wherein each said retaining lug has a length greater than one half of the internal width of said recessed guard.

In one embodiment of the present invention, wherein the other end of said infusion tube is connected to a connector.

In one embodiment of the present invention, wherein the connector is provided with a transfixion needle connected to the infusion tube, wherein the length of the connector is longer than the length of the transfixion needle.

### BRIEF DESCRIPTION OF THE DRAWINGS

FIG. 1 is a schematic elevational view of a intravenous needle set according to the prior art.

FIG. 2 is a schematic elevational view of a safety intravenous needle set in an opened status in accordance with a first embodiment of the present invention.

FIG. 3 corresponds to FIG. 2, illustrating the safety intravenous needle set in a closed status.

FIG. 4 is a bottom view of a part of the safety intravenous needle set shown in FIG. 3.

FIG. 4A is an enlarged view of a part of FIG. 4.

FIG. 5 is a cross sectional view, in an enlarged scale, of a part of the safety intravenous needle set shown in FIG. 3.

FIG. 6 is a schematic cross sectional view of a safety intravenous needle set in a closed status in accordance with a second embodiment of the present invention.

FIG. 7 is a schematic elevational view of a safety intravenous needle set in accordance with a third embodiment of the present invention.

FIG. 8 is a bottom view of a part of a safety intravenous needle set in accordance with a fourth embodiment of the present invention.

FIG. 8A is an enlarged view of a part of FIG. 8.

FIG. 9 is a schematic cross sectional view, in an enlarged scale, of the safety device of the safety intravenous needle set in accordance with the fourth embodiment of the present invention.

FIG. 10 corresponds to FIG. 8, illustrating an alternate form of the arrangement of the retaining lugs in the recessed guard of the safety device.

FIG. 10A is an enlarged view of a part of FIG. 10.

FIG. 11 illustrates a safety intravenous needle set in accordance with a fifth embodiment of the present invention.

FIG. 12 illustrates an alternate form of the anti-slip structure of the safety device of the safety intravenous needle set in accordance with the fifth embodiment of the present invention.

FIG. 13 illustrates another alternate form of the anti-slip structure of the safety device of the safety intravenous needle set in accordance with the present invention.

FIG. 14 illustrates another alternate form of the anti-slip structure of the safety device of the safety intravenous needle set in accordance with the present invention.

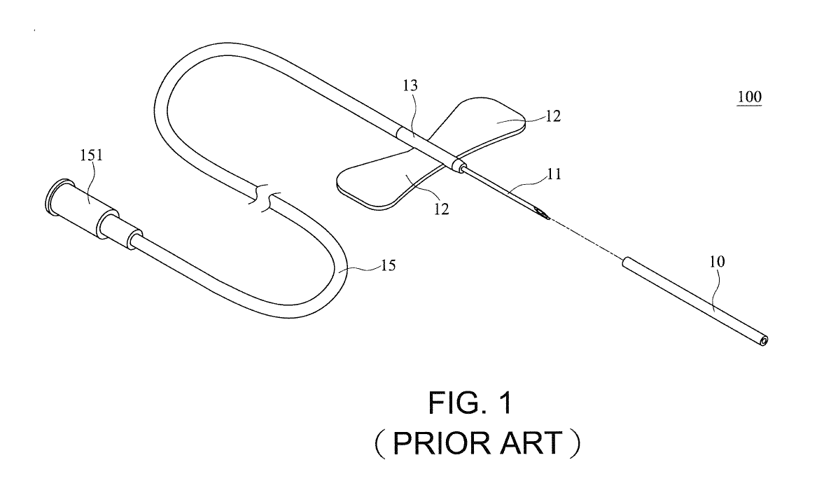

### DETAILED DESCRIPTION OF THE PREFERRED EMBODIMENTS

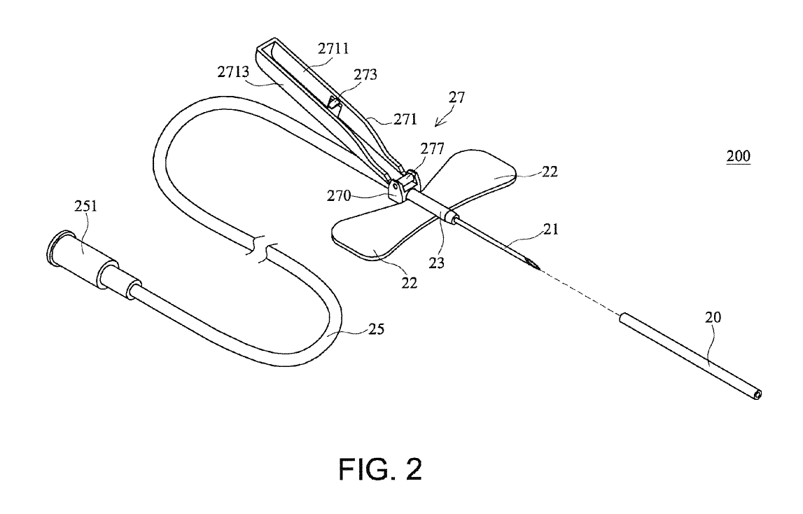

Please refer to FIGS. 2 through 5, a schematic elevational view of a safety intravenous needle set in an opened status in accordance with a first embodiment of the present invention, a schematic elevational view of the safety intravenous needle set in a closed status, a bottom view of a part of the safety intravenous needle set, an enlarged view of a part of the safety intravenous needle set and a sectional enlarged view of a part of the safety needle set are shown. As illustrated, the safety intravenous needle set 200 comprises a medical needle 21, a needle hub 23, an infusion tube 25, and a safety device 27.

The needle hub 23 has its one end connected with the medical needle 21 and its other end connected with the infusion tube 25. Further, the needle hub 23 has two wings 22 arranged at two opposite lateral sides thereof. Further, a needle cap 20 is detachably capped on the medical needle 21 for protection.

The infusion tube 25 is a fluid passage for intravenous infusion or drawing blood, having its one end connected to the needle hub 23 and its other end provided with a connector 251 for the connection of a feeding tube of an infusion bag, saline bag or blood bag, or a medical device.

The safety device 27 is an anti-piercing device, comprising a base block 270, a recessed guard 271, and a pivot 277. The recessed guard 271 comprises two inner walls 2711, an outer wall 2713, and at least one retaining lug 273 located on one inner wall 2711. The base block 270 is mounted at the needle hub 23. The recessed guard 271 is pivotally connected to the base block 270 by the pivot 277. By means of the pivot 277, the recessed guard 271 is biasable relative to the base block 270.

Before the use of the safety intravenous needle set 200, as shown in FIG. 2, the safety device 27 is opened from the medical needle 21. When using the safety intravenous needle set 200, the healthcare worker can remove the needle cap 20 from the medical needle 21 to expose the medical needle 21. By means of the wings 22, the healthcare worker can conveniently insert the medical needle 21 into the patient's vein, blood vessel or body and then secure the wings 22 to the patient's skin by a medical adhesive tape. At this time, the healthcare worker can start an intravenous infusion or blood draw.

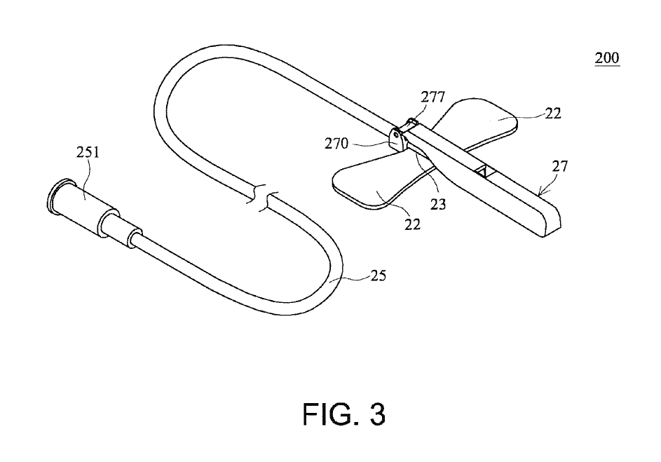

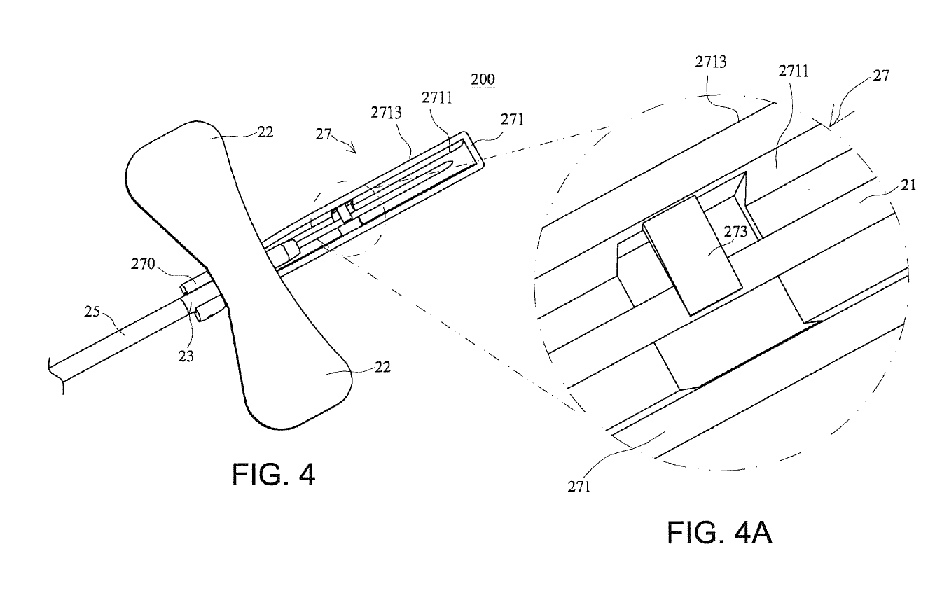

After the service of the safety intravenous needle set 200, the healthcare worker will remove the medical needle 21 from the body of the patient. As illustrated in FIG. 3, FIG. 4 and FIG. 4A, when going to dispose of the used medical needle 21, the healthcare worker can turn the recessed guard 271 about the pivot 277 to the top side of the medical needle 21 and then press down the recessed guard 271 or push the medical needle 21 upwardly, forcing the medical needle 21 into the inside of the recessed guard 271. At this time, the medical needle 21 is secured inside the recessed guard 271 by the retaining lug 273. Thus, the safety device 27 and the medical needle 21 are secured together in the closed status, and the used medical needle 21 is well protected inside the safety device 27 for further medical waste treatment.

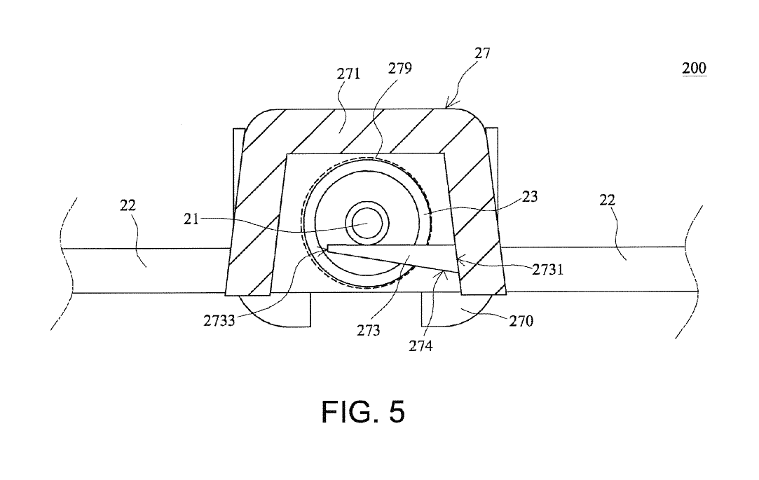

Referring to FIG. 5, the retaining lug 273 has opposing connection end 2731 and retaining end 2733. By means of the connection end 2731, the retaining lug 273 is affixed to one inner wall 2711 of the recessed lug 271. The connection end 2731 has a relatively larger thickness than the retaining end 2733. Further, the retaining lug 273A defines a bevel face 274 on one side thereof between the connection end 2731 and the retaining end 2733. Subject to the arrangement of the bevel face 274, the medical needle 21 can be moved along the bevel face 274 into the inside of the recessed guard 271 easily. When pushed by an external pressure, the medical needle 21 can be easily moved into the gap between the retaining lug 273 and the recessed guard 271 with less effort.

In one embodiment of the present invention, the base block 270 of the safety device 27 comprises a hollow passage 279 (see the imaginary line in FIG. 5), or a C-shaped ring, for the insertion and affixation of the needle hub 23 of the safety intravenous needle set 200 to have the base block 270 be mounted at the needle hub 23.

Further, if only one single retaining lug 273 is provided, the length of the retaining lug 273 should be greater than one half of the internal width of the recessed guard 271, i.e., the retaining end 2733 will extend across the middle line of the recessed guard 271 where the medical needle 21 is to be accommodated. When forcing the medical needle 21 toward the inside of the recessed guard 271, the retaining lug 273 will be forced to curve inwardly by the medical needle 21, and the medical needle 21 will be forced to displace sideways and then to enter the gap between the retaining lug 273 and the recessed guard 271.

After the medical needle 21 entered the recessed guard 271, the medical needle 21 and the retaining lug 273 immediately return to their former position or shape so that the medical needle 21 can be positively positioned in the middle position inside the recessed guard 271 and the retaining end 2733 of the retaining lug 273 can transversely extend over the medical needle 21. Under normal impact or external pressure, the medical needle 21 will not be forced to displace sideways or to move out of the recessed guard 271. Thus, the medical needle 21 can be well accommodated after its service, avoiding a repeat use.

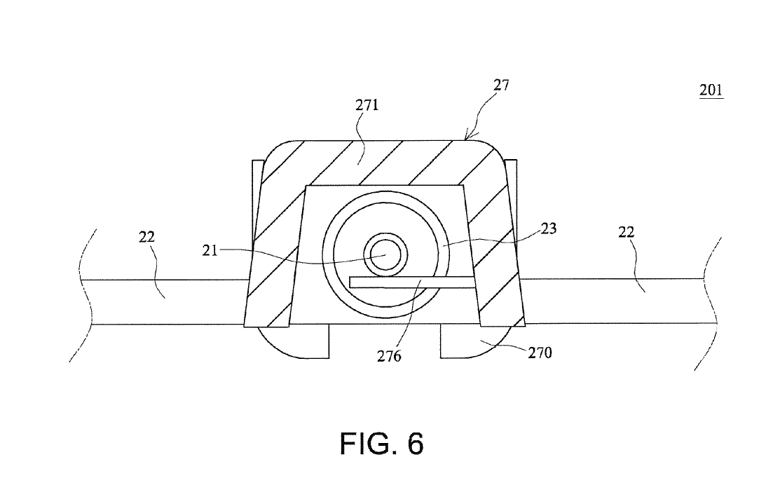

FIG. 6 illustrates a safety intravenous needle set 201 in accordance with a second embodiment of the present invention. According to this second embodiment, the retaining lug 276 is a planar thin sheet member without the aforesaid bevel face (274). This planar thin sheet member of retaining lug 276 is elastically and inwardly bendable so that the medial needle 21 can be moved along the elastically and inwardly deformed curvature of the retaining lug 276 into the inside of the recessed guard 271.

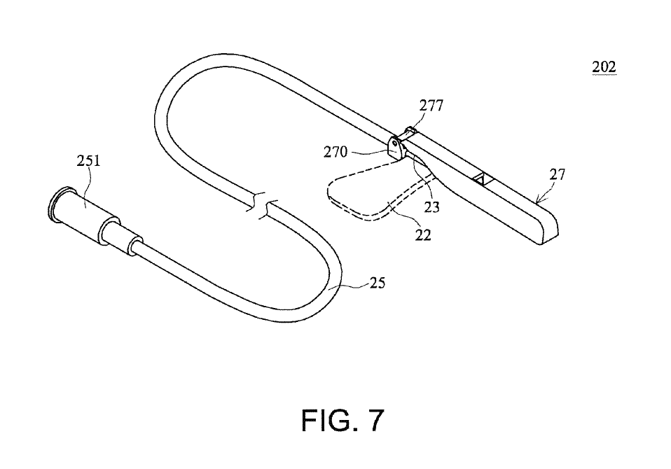

Further, as shown in FIG. 7, illustrates a safety intravenous needle set 202 in accordance with a third embodiment of the present invention. According to this third embodiment, the needle hub 23 has only one wing 22 located on one lateral side thereof. The other lateral side of the needle hub 23 is kept clean. In order to facilitate the healthcare worker to manipulate the safety intravenous needle set 202, the thickness of the single wing 22 shall be relatively thicker that the double wing design in the aforesaid first and second embodiments.

It is to be understood that the needle hub 23 can be made without the wing 22. A safety intravenous needle set 202 without the wing 22 saves much the manufacturing cost and is applicable for use as a regular injection syringe needle, for example, intramuscular injection syringe needle.

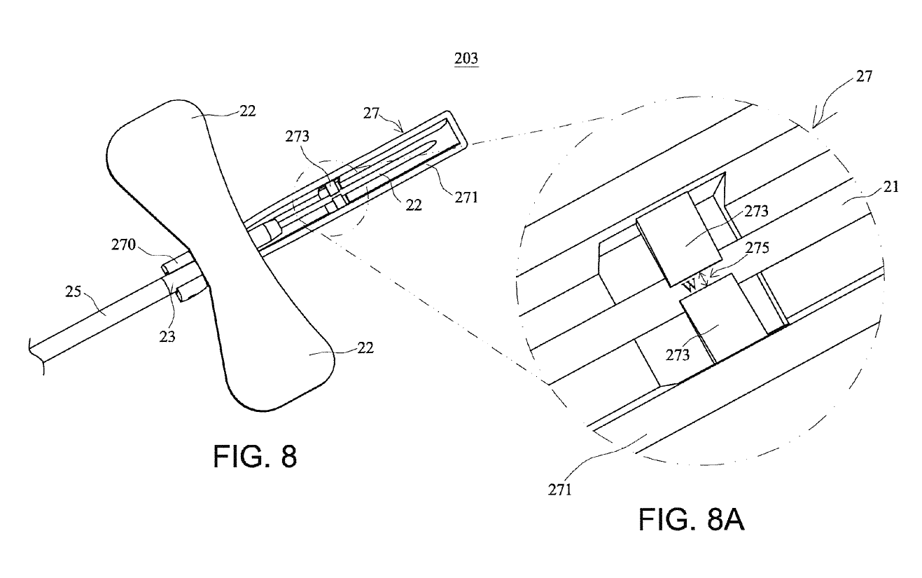

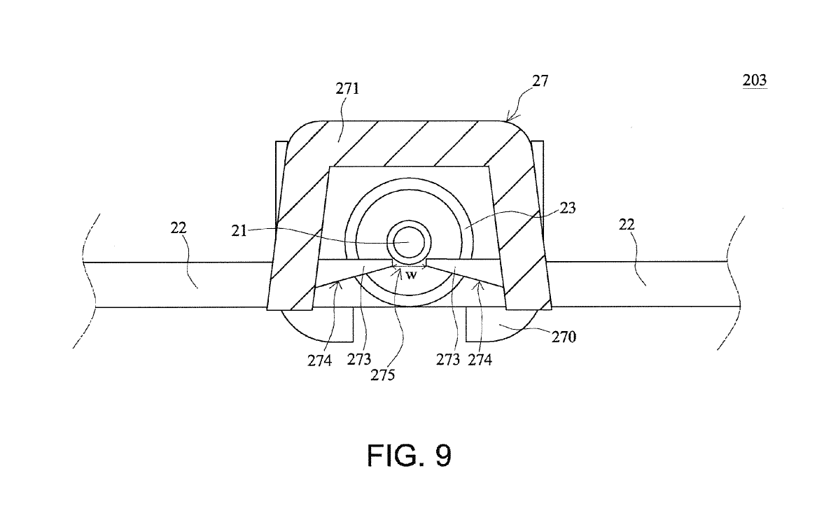

FIG. 8, FIG. 8A and FIG. 9 illustrate a safety intravenous needle set 203 in accordance with a fourth embodiment of the present invention. In the aforesaid first, second or third embodiment, the recessed guard has one retaining lug 273 or 276 located on only one inner wall thereof. According to this fourth embodiment, the recessed guard 271 has at least one retaining lug 273 or 276 located on each of the two inner walls thereof. Each retaining lug 273 or 276 can be configured to provide a bevel face 274, or in the shape of a planar thin sheet member.

Further, a constraint gap 275 is defined between the two retaining lugs 273 or 276 (or each pair of retaining lugs) that are symmetrically arranged at the two inner walls of the recessed guard 27. The constraint gap 275 has a width W approximately equal to the diameter of the medical needle 21 so that the medical needle 21 cannot be easily forced out of the recessed guard 27 through the constraint gap 275. Preferably, the width W of the constraint gap 275 is shorter than the diameter of the medical needle 21.

When the healthcare worker is going to recover the used medical needle 21, bias the recessed guard 271 of the safety device 27 toward the medical needle 21, and then force the medical needle 21 through the constraint gap 275 into the inside of the recessed guard 271, enabling the used medical needle 21 to be held inside the recessed guard 271 by the retaining lugs 273. Thus, the retaining lugs 273 hold down the used medical needle 21 inside the recessed guard 271, avoiding exposure of the used medical needle 21 to the outside of the recessed guard 271 or a repeated use of the safety intravenous needle set 203.

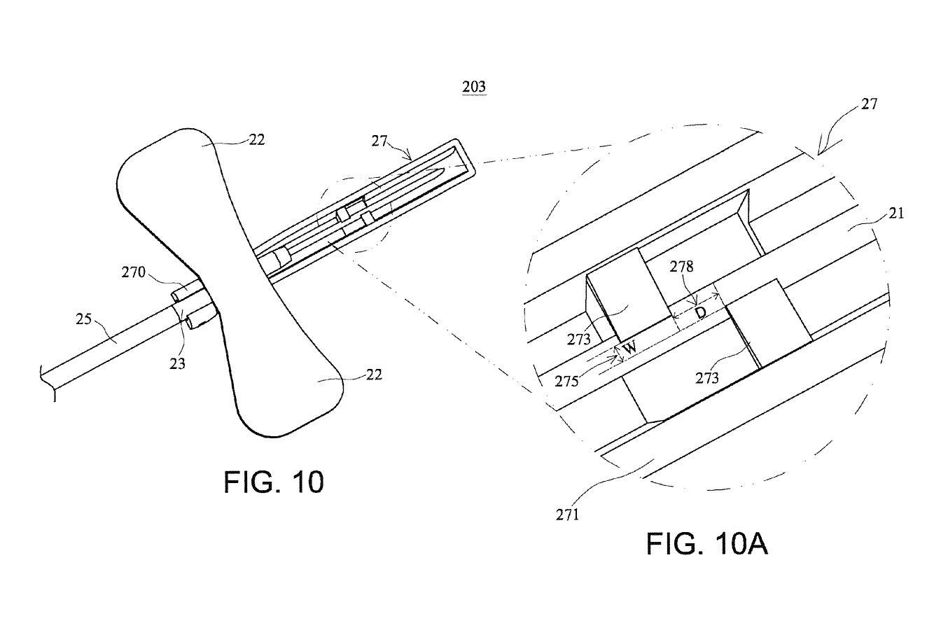

Further, as shown in FIG. 10 and FIG. 10A, the retaining lugs 273 of the safety intravenous needle set 203, unlike the symmetrical arrangement shown in FIG. 8, can be arranged in a staggered manner with a longitudinal pitch (D) 278 defined therebetween. In this case, one retaining lug 273 can retain a front part of the medical needle 21 and the other retaining lug 273 can retain a rear part of the medical needle 21, enhancing the positioning stability of medical needle 21 in the recessed guard 27.

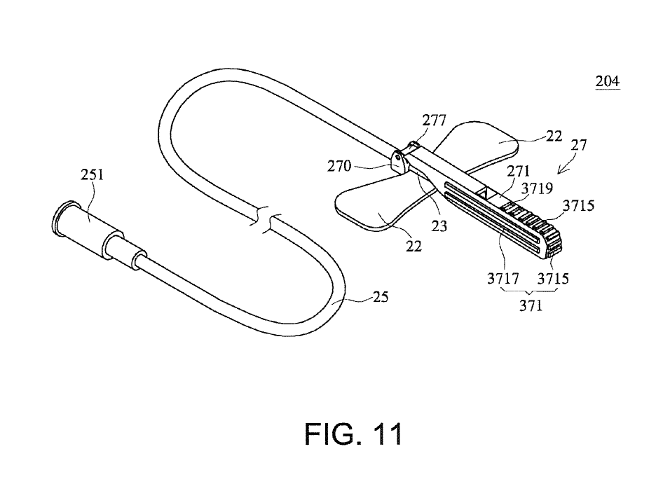

FIG. 11 illustrates a safety intravenous needle set 204 in accordance with a fifth embodiment of the present invention. After the service of the safety intravenous needle set 204, the healthcare worker normally will bias the recessed guard 271 of the safety device 27 toward the medical needle 21. At this time, improper posture or improper finger positioning on the recessed guard 271 may cause the user's fingers to slip over the recessed guard 271 and to be injured by the medical needle 21. To avoid this problem, an anti-slip structure 371 is located on the outer wall 2713 of the recessed guard 271. According to this embodiment, the anti-slip structure 371 comprises a plurality of transverse ribs 3715 located on the top side of the outer wall 2713 of the recessed guard 271, and a plurality of longitudinal ribs 3717 located on two opposite lateral sides of the outer wall 2713.

When the healthcare worker or any other user is going to dispose of the used medical needle 21, hold the outer wall 2713 with the fingers and then bias the recessed guard 271 toward the medical needle 21. Subject to the anti-slip effect of the anti-slip structure 371, the healthcare worker or user can hold the outer wall 2713 of the recessed guard 271 with the fingers positively, avoiding finger slipping or accidental needle stick injury.

It is to be understood that the anti-slip structure 371 is not limited to the transverse ribs 3715 and/or longitudinal ribs 3717. Alternatively, the anti-slip structure 371 can be formed of raised portions, or longitudinally and/or transversely extending grooves 3719, achieving the safe anti-slip effect.

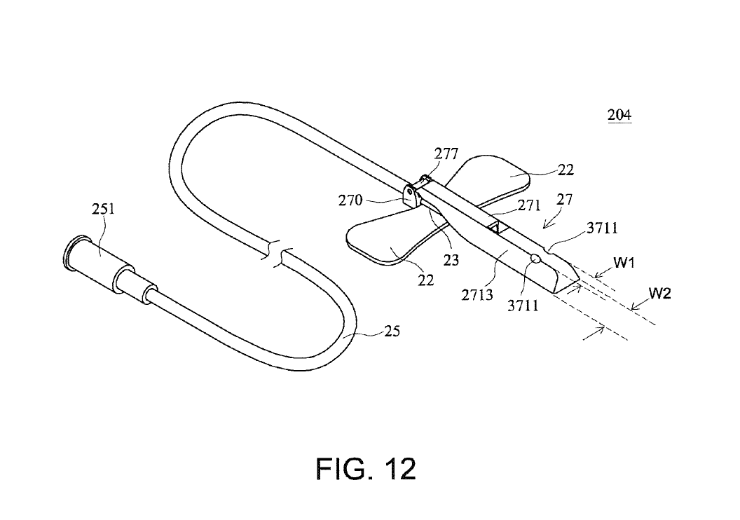

Further, as shown in FIG. 12, the anti-slip structure of the safety device 27 of the safety intravenous needle set 204 can comprise at least one finger notch 3711 on the outer wall 2713. When biasing the safety device 27, the healthcare worker or user can rest the thumb and/or forefinger in the at least one finger notch 3711 of the recessed guard 271 and then bias the recessed guard 271.

As the user's thumb and/or forefinger is (are) rested in the at least one finger notch 3711 of the recessed guard 271 when holding the recessed guard 271, the user's thumb and/or forefinger is (are) prohibited from displacement relative to the recessed guard 271, avoiding accidental needle stick injury.

Further, the width W1 of the top side and the width W2 of the bottom side of the recessed guard 271 are unequal. The width W2 of the bottom side of the recessed guard 271 is greater than the width W1 of the top side of the recessed guard 271. Thus, the two opposite lateral sides of the outer wall 2713 of the recessed guard 271 of the safety device 27 provide one respective bevel face. If the user's fingers slip away when moving the recessed guard 271 of the safety device 27, the bevel faces of the outer wall 2713 of the recessed guard 271 guide the user's fingers away from the effective range of the medical needle 21, assuring a high level of safety of the use of the safety intravenous needle set 204.

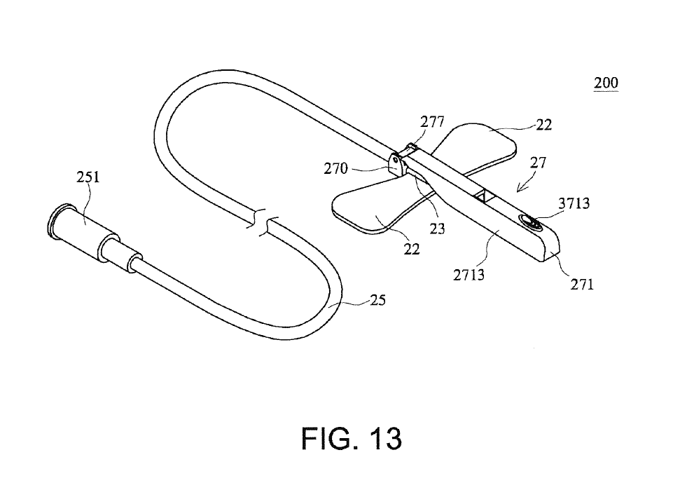

Further, FIG. 13 illustrates another alternate form of the anti-slip structure. According to this embodiment, the anti-slip structure comprises a finger retaining means 3713 located on the outer wall 2713 of the recessed guard 271 of the safety device 27 of the safety intravenous needle set 200. The finger retaining means 3713 can be a recessed portion or raised portion with ribs or grooves therein. The anti-slip structure 3713 can prohibit the user's fingers from displacement relative to the recessed guard 271, avoiding accidental needle stick injury.

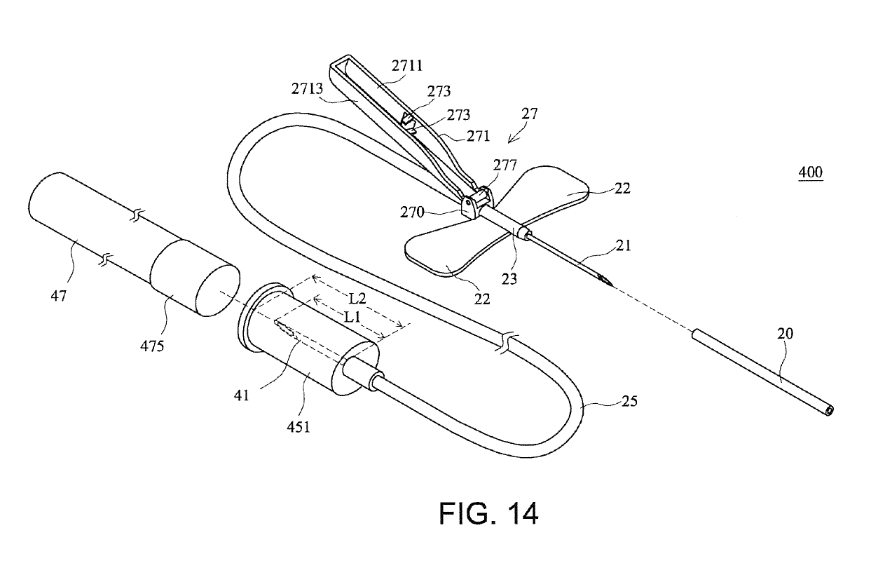

FIG. 14 illustrates a safety intravenous needle set in accordance with one embodiment of the present invention. The safety intravenous needle set 400 of the present invention can be used for blood lancing device or a safety infusion needle set. In the present embodiment, one end of the infusion tube 25 is connected to the needle hub 23. the other end of the infusion tube 25 is connected to a connector 45L The connector 451 is provided with a transfixion needle 41 for piercing a stopper 475 of a blood collection tube 47. That the blood can flow into the blood collection tube 47 through the medical needle 21, the infusion tube 25, and the transfixion needle 41.

When the blood collection tube 47 is filled with enough blood or body fluid, the blood collection tube 47 is pulled out of the connector 451. Since the length L2 of the connector 451 is longer than the length L1 of the transfixion needle 41, the transfixion needle 41 with blood or body fluid is retained in the connector 451 for avoiding accidental needle stick injury. The medical needle 21 at the front of the safety intravenous needle set 400 is also secured by the safety device 27.

In conclusion, the invention provides a safety intravenous needle set 200/201/202/203/204 equipped with a safety device 27. After the service of the medical needle 21, the recessed guard 271 of the safety device 27 can be biased to the medical needle 21 to let the medical needle 21 be forced into the inside of the recessed guard 271 and secured therein, avoiding accidental needle stick injury or a repeat use of the safety intravenous needle set and assuring a high level of medical waste disposal safety.

The foregoing description is merely one embodiment of the present invention and not considered as restrictive. All equivalent variations and modifications in shape, structure, feature, and spirit in accordance with the appended claims may be made without in any way from the scope of the invention.

What is claimed is:

1. A safety intravenous needle set, comprising:
a needle hub holding a medical needle at one end thereof and an infusion tube at an opposite end thereof; and
a safety device, said safety device comprising a base block, a recessed guard and a pivot, said recessed guard comprises two opposing inner walls, an outer wall and at least one pair of retaining lugs located on said two opposing inner walls, each said pair of retaining lugs defining therebetween a constraint gap, said base block being fixedly mounted at said needle hub, said recessed guard being pivotally connected to said base block by said pivot for allowing said recessed guard to be biased relative to said base block and said needle hub toward said medical needle to let said medical needle be forced through said constraint gap into the inside of said recessed guard.

2. The safety intravenous needle set as claimed in claim 1, wherein said recessed guard has a relatively wider top side and a relatively narrower bottom side so that said outer wall defines a bevel face at each of two opposite lateral sides thereof.

3. The safety intravenous needle set as claimed in claim 1, wherein said recessed guard comprises an anti-slip structure located on said outer wall.

4. The safety intravenous needle set as claimed in claim 3, wherein said anti-slip structure comprises at least one longitudinal rib, at least one transverse rib, at least one raised portion, at least one recessed portion, at least one finger notch and/or at least one finger retaining means.

5. The safety intravenous needle set as claimed in claim 1, wherein said needle hub comprises a wing located on at least one of two opposite lateral sides thereof.

6. The safety intravenous needle set as claimed in claim 1, wherein each said retaining lug is configured to provide a bevel face, or in the shape of a planar thin sheet member.

7. The safety intravenous needle set as claimed in claim 1, wherein said retaining lugs of said safety device are arranged in a staggered manner, defining a longitudinal pitch between each two adjacent said retaining lugs.

8. The safety intravenous needle set as claimed in claim 1, wherein said constraint gap has a width equal to or smaller than the diameter of said medical needle.

9. The safety intravenous needle set as claimed in claim 1, wherein the other end of said infusion tube is connected to a connector.

10. The safety intravenous needle set as claimed in claim 9, wherein the connector is provided with a transfixion needle connected to the infusion tube, wherein the length of the connector is longer than the length of the transfixion needle.

11. A safety intravenous needle set, comprising:
a needle hub holding a medical needle at one end thereof and an infusion tube at an opposite end thereof; and
a safety device, said safety device comprising a base block, a recessed guard and a pivot, said recessed guard comprises two opposing inner walls, an outer wall and at least one retaining lug located on one said inner wall, said base block being fixedly mounted at said needle hub, said recessed guard being pivotally connected to said base block by said pivot for allowing said recessed guard to be biased relative to said base block and said needle hub toward said medical needle to let said medical needle be forced over said at least one retaining lug into the inside of said recessed guard and then retaining inside said recessed guard by said at least one retaining lug.

12. The safety intravenous needle set as claimed in claim 11, wherein said recessed guard has a relatively wider top side and a relatively narrower bottom side so that said outer wall defines a bevel face at each of two opposite lateral sides thereof.

13. The safety intravenous needle set as claimed in claim 11, wherein said recessed guard comprises an anti-slip structure located on said outer wall.

14. The safety intravenous needle set as claimed in claim 13, wherein said anti-slip structure comprises at least one longitudinal rib, at least one transverse rib, at least one raised portion, at least one recessed portion, at least one finger notch and/or at least one finger retaining means.

15. The safety intravenous needle set as claimed in claim 11, wherein said needle hub comprises a wing located on at least one of two opposite lateral sides thereof.

16. The safety intravenous needle set as claimed in claim 11, wherein each said retaining lug is configured to provide a bevel face, or in the shape of a planar thin sheet member.

17. The safety intravenous needle set as claimed in claim 11, wherein each said retaining lug has a length greater than one half of the internal width of said recessed guard.

18. The safety intravenous needle set as claimed in claim 11, wherein the other end of said infusion tube is connected to a connector.

19. The safety intravenous needle set as claimed in claim 18, wherein the connector is provided with a transfixion needle connected to the infusion tube, wherein the length of the connector is longer than the length of the transfixion needle.
